(19) United States
(12) Patent Application Publication
(10) Pub. No.: US 2023/0264039 A1
(43) Pub. Date: Aug. 24, 2023

(54) LIGHT THERAPY DEVICE WITH FUNCTION OF RELEASING NEGATIVE ION

(71) Applicants: Po-Chang LIU, TAOYUAN CITY (TW); BIBOTING INTERNATIONAL CO., LTD., Taoyuan City (TW)

(72) Inventors: Po-Chang LIU, TAOYUAN CITY (TW); Chia-Hsueh HSIEH, TAOYUAN CITY (TW)

(21) Appl. No.: 17/676,776

(22) Filed: Feb. 21, 2022

(51) Int. Cl. A61N 5/06; A61M 1/08; A61M 35/00

(52) U.S. Cl. CPC A61N 5/0616; A61M 1/08; A61M 35/10; A61M 2205/051; A61N 2005/0649; A61N 2005/0651; A61N 2005/0663

(57) ABSTRACT

A light therapy device used for a negative-pressure cup is provided. The light therapy device includes a carrier, a light therapy assembly, a negative ion assembly and a needle protection cap. The carrier is disposed in the negative-pressure cup and includes a panel. The light therapy assembly and the negative ion assembly are disposed in the carrier. The negative ion assembly includes a releasing needle. The releasing needle projects out from the panel to release negative ions. The needle protection cap covers the panel corresponding to the releasing needle to shield the releasing needle. Therefore, the light therapy assembly and the negative ion assembly may be associated with the negative-pressure cup for usage, and the needle protection cap may prevent the user from being stabbed.

### BACKGROUND

### Technical Field

The disclosure relates to a light therapy device, and in particularly, to a light therapy device with a function of releasing negative ion.

### Description of Related Art

A light therapy device utilizes a light emitting unit to emit light which can relax uncomfortable part of a human body for light therapy.

The inventor of the disclosure studies to associate a light therapy device and a negative ion device with a negative-pressure cup, so that a surface of the uncomfortable part of a patient's body can be sucked to form a bulge by sucking air in the cup to form a negative-pressure status in the negative-pressure cup, meanwhile, the light therapy device and the negative ion device can perform light therapy and negative ion attachment to the bulge. Thus, it has a better relaxation effect to the patient's uncomfortable part.

However, the negative ion device must use a releasing needle to release negative ions, and the bulge on the patient's uncomfortable part, which is formed by suction of the negative-pressure cup, protrudes toward the releasing needle. Experiments have confirmed that the bulge is often stabbed by the releasing needle, which may cause the risk of infection.

Therefore, how to design a combination of light therapy, negative ions, and a negative-pressure cup with preventing a patient's bulge from being stabbed by a releasing needle is an important issue.

In view of this, the inventors have devoted themselves to the above-mentioned related art, researched intensively and cooperated with the application of science to try to solve the above-mentioned problems.

### SUMMARY

An object of the disclosure is to provide a light therapy device with a function of releasing negative ion, which may combine a light therapy assembly and a negative ion assembly with a negative-pressure cup for usage, and use a needle protection cap to prevent a patient's bulge from being stabbed by a releasing needle.

To accomplish the above object, the disclosure provides a light therapy device with a function of releasing negative ion and used for a negative-pressure cup. The light therapy device includes a carrier, a light therapy assembly, a negative ion assembly and a needle protection cap. The carrier is disposed in the negative-pressure cup and includes a panel. The light therapy assembly is disposed in the carrier. The negative ion assembly is disposed in the carrier and includes a releasing needle. The releasing needle projects out from the panel to release negative ions. The needle protection cap covers the panel corresponding to the releasing needle to shield the releasing needle.

In comparison with the related art, the disclosure has the following functions. The light therapy assembly and the negative ion assembly may be incorporated with the negative-pressure cup, and a needle protection cap is used to prevent a patient's bulge from being stabbed by a releasing needle.

### BRIEF DESCRIPTION OF THE DRAWINGS

FIG. 1 is an exploded view of the light therapy device of the disclosure at a top point of view;

FIG. 2 is an exploded view of the light therapy device of the disclosure at a bottom point of view;

FIG. 3 is an assembled view of the light therapy device of the disclosure according to FIG. 2;

FIG. 4A is a cross-sectional view of the light therapy device of the disclosure according to FIG. 3 (without the negative ion releasing head);

FIG. 4B is a cross-sectional view of the light therapy device of the disclosure according to FIG. 3 (with the negative ion releasing head);

FIG. 5 is an exploded view of the light therapy device of the disclosure before being installed to the negative-pressure cup;

FIG. 6 is an exploded view of the light therapy device of the disclosure after being installed to the negative-pressure cup; and

FIG. 7 is a cross-sectional view of the light therapy device of the disclosure according to FIG. 6.

### DETAILED DESCRIPTION

The technical contents of this disclosure will become apparent with the detailed description of embodiments accompanied with the illustration of related drawings as follows. It is intended that the embodiments and drawings disclosed herein are to be considered illustrative rather than restrictive.

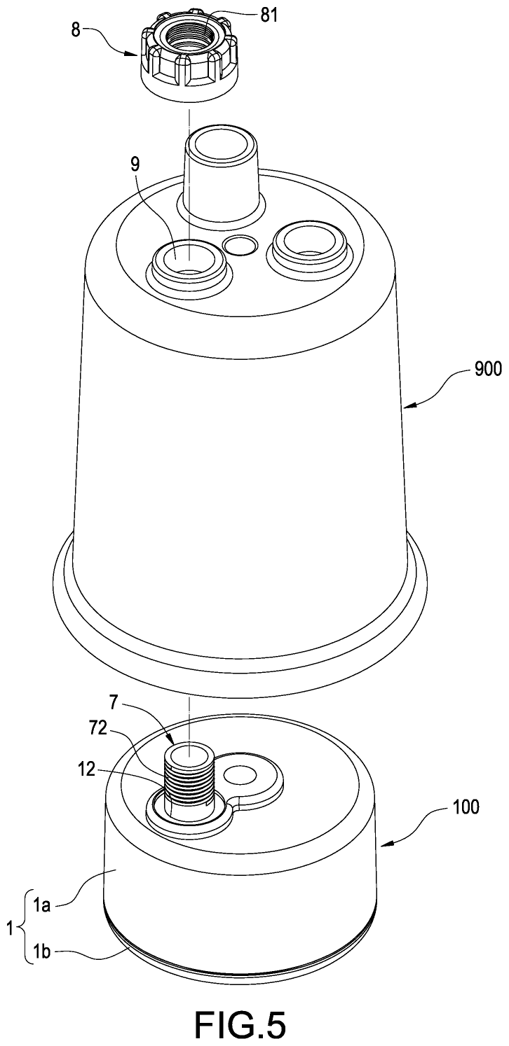

The disclosure provides a light therapy device with a function of releasing negative ion, as shown in FIG. 5, which is used for associating with a negative-pressure cup 900, for functions of both light therapy and releasing negative ion.

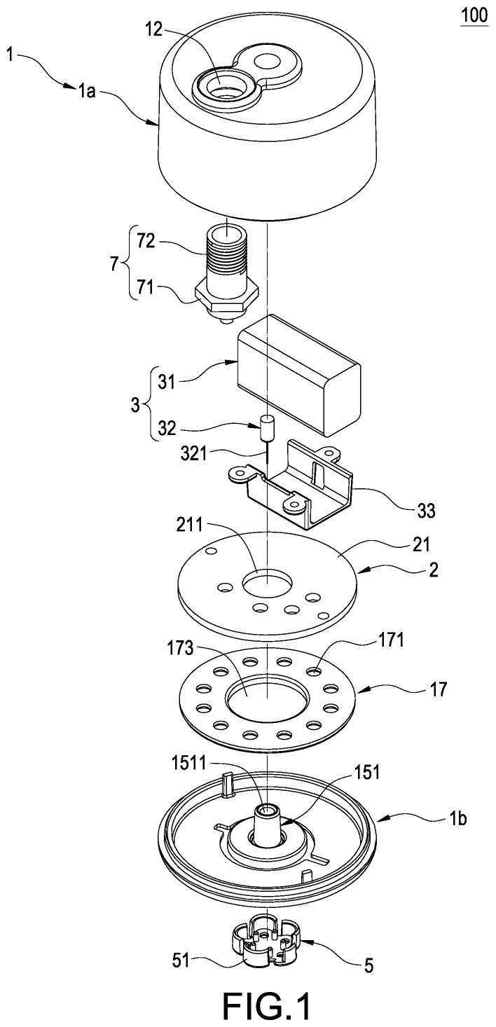

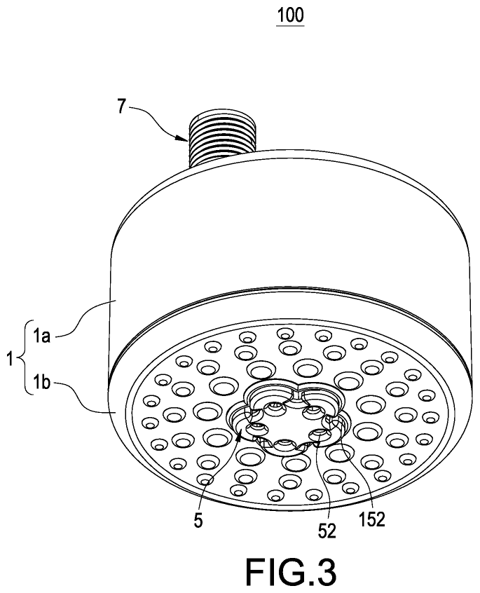

As shown in FIGS. 1-5, the light therapy device with a function of releasing negative ion of the disclosure (hereinafter “light therapy device 100”) includes a carrier 1, a light therapy assembly 2, a negative ion assembly 3 and a needle protection cap 5. In some embodiments, the disclosure further includes a fixing rod 7 and a fixing member 8.

The carrier 1 may be any structure capable of being disposed in the negative-pressure cup 900 and have a panel 15 in a light-permeable manner. The shown embodiment uses the carrier 1 having a base 1a and a light-permeable cover 1b as an example. The base 1a and the light-permeable cover 1b may be assembled with each other.

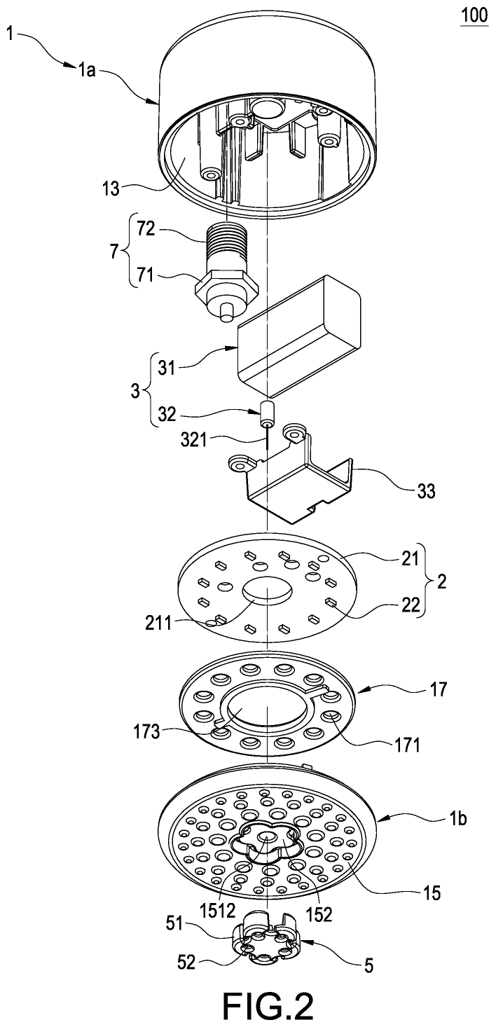

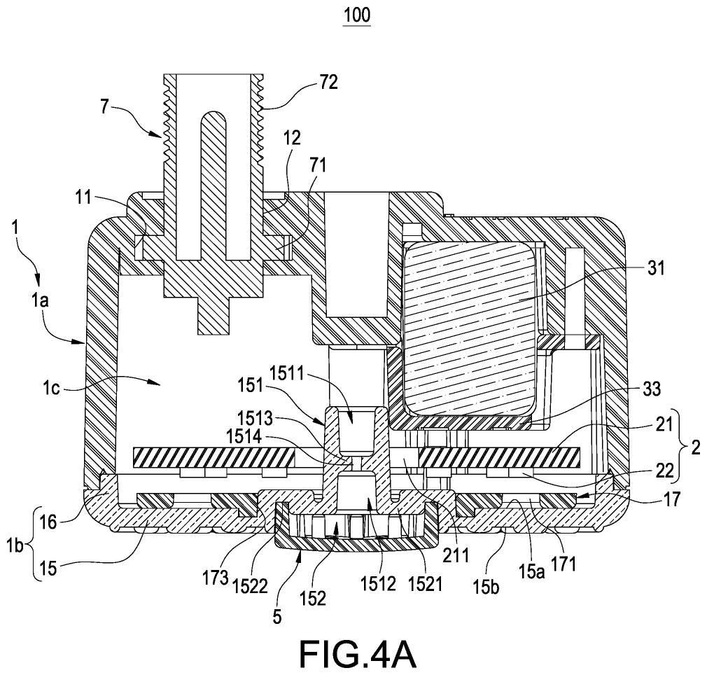

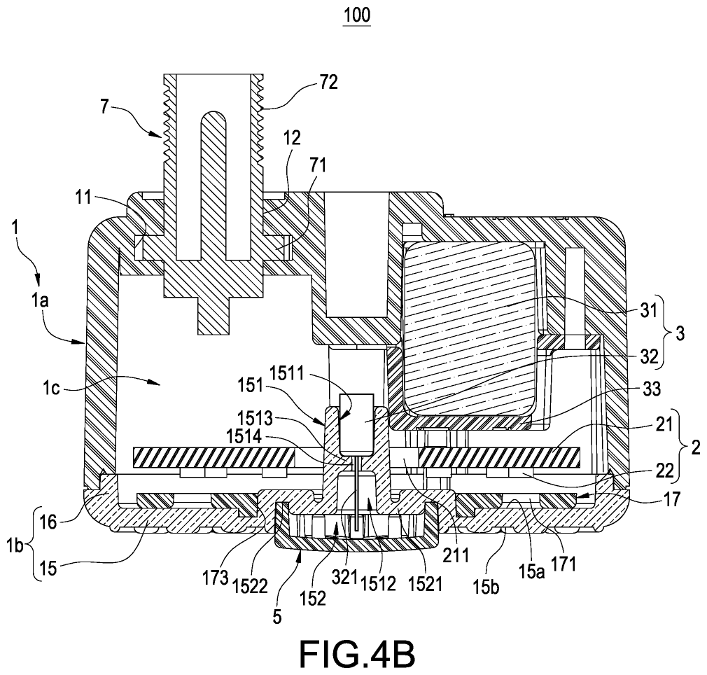

The base 1a has an opening 13 as shown in FIG. 2. The light-permeable cover 1b covers the base 1a corresponding to position of the opening 13 to assembled with each other, and a receiving space 1c is jointly formed between the base 1a and the light-permeable cover 1b as shown in FIG. 4A. The light-permeable cover 1b has the light-permeable panel 15.

The light therapy assembly 2 is disposed in the receiving space 1c of the carrier 1 for emitting light via the light-permeable cover 1b (or the panel 15). A patient's uncomfortable part may be relaxed (light therapy) after being irradiated by the light.

The negative ion assembly 3 is also disposed in the receiving space 1c. The negative ion assembly 3 has a releasing needle 321 which projects outside the panel 15 (also outside the carrier 1) to release negative ions.

The needle protection cap 5 covers the panel 15 corresponding to the releasing needle 321 (or position of a needle receiving recess 1512) to make the releasing needle 321 be shielded by the needle protection cap 5 as shown in FIG. 4B.

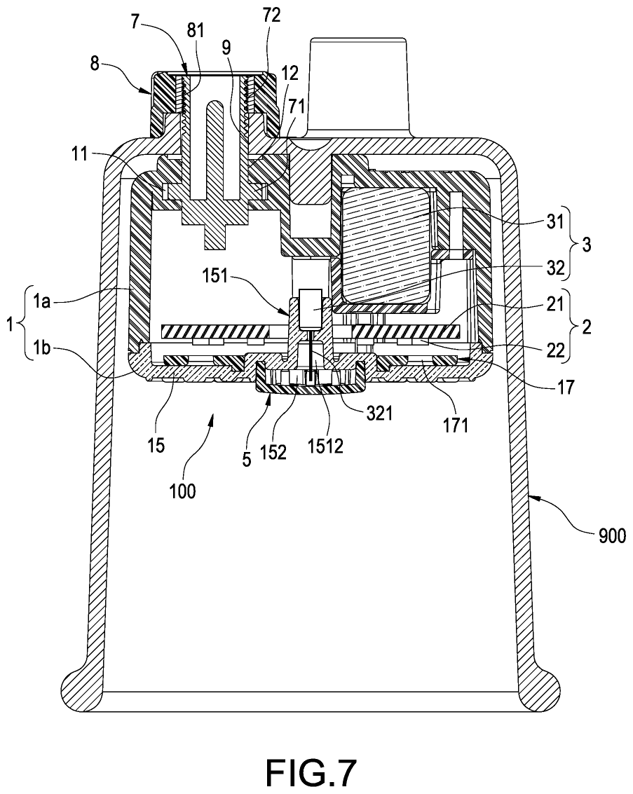

As shown in FIG. 7, when the light therapy device 100 is assembled in the negative-pressure cup 900, the air in the cup is sucked to form a negative-pressure status in the negative-pressure cup 900 to suck a patient's skin to form a bulge (not shown in figures). Meanwhile, the light therapy device 100 may further use the light therapy assembly 2 to perform light therapy to the bulge and use the releasing needle 321 of the negative ion assembly 3 to release negative ions to the bulge. Also, the disclosure uses the needle protection cap 5 to cover the releasing needle 321, so that the bulge may not be stabbed by the releasing needle 321 and a protective effect for stabbing injury may be achieved.

As shown in FIGS. 1-4, in detail, the light-permeable cover 1b includes a ring 16 and the panel 15. The ring 16 surrounds a periphery of the panel 15. The light-permeable cover 1b is connected to the base 1a by the ring 16. The panel 15 has an inner side 15a and an outer side 15b opposite to each other. The panel 15 is dented from the outer side 15b toward the inner side 15a to form a needle receiving recess 1512. In some embodiments, the panel 15 is further dented to form a receiving cavity 1511. The receiving cavity 1511 and the needle receiving recess 1512 are separated by a first bottom plate 1513. The first bottom plate 1513 is formed with a through hole 1514. It is noted that the panel 15 is integratedly extended with a post 151 of hollow shape toward the receiving space 1c, and the first bottom plate 1513 is located at the middle of the post 151 for separation so that the receiving cavity 1511 and the needle receiving recess 1512 are formed in the post 151 with a back-to-back manner.

In some embodiments, the panel 15 is further dented with a cap receiving recess 152 toward the same direction with the same manner of the needle receiving recess 1512. The cap receiving recess 152 has a second bottom plate 1521. The receiving cavity 1511 and the needle receiving recess 1512 are dented from the second bottom plate 1521 to make the panel 15, the second bottom plate 1521 and the first bottom plate 1513 form a step shape from the outside to the inside.

The light therapy assembly 2 includes a circuit board 21 and multiple light emitting elements 22 arranged on the circuit board 21. The light emitting element 22 is a light emitting diode (LED) which has a light therapy effect and a wavelength between 415±10 nm and 830±10 nm. For example, the light emitting element 22 may be a green LED with a wavelength of 525±15 nm or 525±30 nm. The light therapy assembly 2 is formed with an escape hole 211 and arranged to be parallel with the panel 15 at an interval so that the post 151 passes through the escape hole 211 and the light therapy assembly 2 emits light toward the panel 15 after the light therapy assembly 2 has been disposed in the carrier 1. The light-permeable cover 1b may further include a shading plate 17 which is assembled on the inner side 15a of the panel 15 with a face-to-face manner. The shading plate 17 is formed with multiple via holes 171 corresponding to each light emitting element 22 so that each light emitting element 22 emits light correspondingly to each via hole 171.

Furthermore, the shading plate 17 is formed with an aperture 173 for being passed by the post 151 and embedded by the protrusion formed by the cap receiving recess 152 dented on the panel 15.

The negative ion assembly 3 includes a negative ion emitter 31 and a negative ion releasing head 32 and may further include a frame 33. The negative ion emitter 31 is fixed in the receiving space 1c by the frame 33. The negative ion releasing head 32 is disposed in the receiving cavity 1511 of the panel 15 and has the releasing needle 321. The releasing needle 321 passes through the through hole 1514 to reach in the needle receiving recess 1512 so that both the negative ion releasing head 32 and the releasing needle 321 may be received in the needle receiving recess 1512.

The needle protection cap 5 has multiple surrounding walls 51 at intervals. The second bottom plate 1521 is formed with an indent 1522 corresponding to the surrounding walls 51. The needle protection cap 5 is fixed in the cap receiving recess 152 and covers the cap receiving recess 152 by embedding the surrounding walls 51 into the indent 1522. As for the manner of fixing the needle protection cap 5 in the cap receiving recess 152, the disclosure does not limit it, for example, it may be that an outer periphery of the needle protection cap 5 tightly abuts against the corresponding inner wall of the cap receiving recess 152, or the surrounding walls 51 are tightly embedded in the indent 1522. The needle protection cap 5 is further formed with multiple passing holes 52 to facilitate spreading negative ions released by the releasing needle 321. The negative ions may also be spread via gaps between every adjacent two of the surrounding walls 51 as shown in FIG. 1 but not labeled.

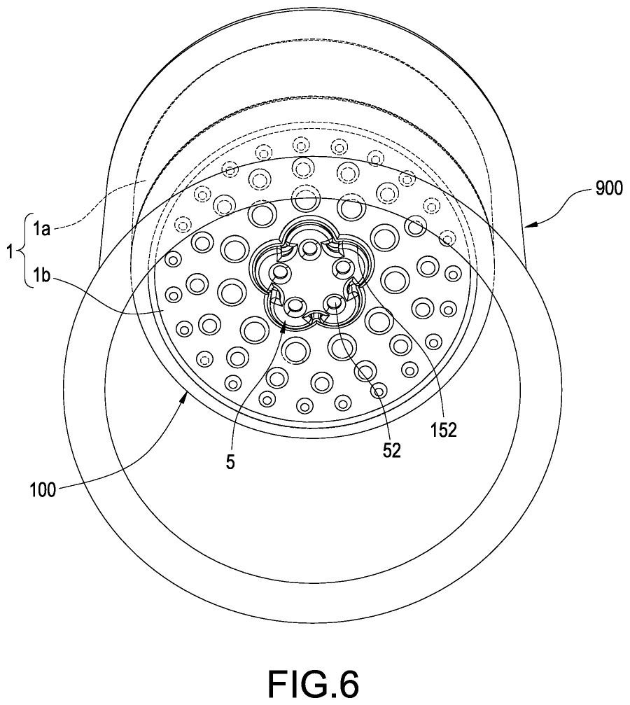

In addition, as shown in FIGS. 1, 2 and 4-7, the structure of the light therapy device 100 of the disclosure, which is used to be disposed in the negative-pressure cup 900, includes a fixing rod 7 and a fixing member 8. The bottom (not labeled) of the negative-pressure cup 900 has multiple penetrating holes 9.

The bottom (not labeled) of the base 1a is formed with a fixing hole 12. The base 1a is further formed with a first corresponding fixing portion 11 corresponding to the fixing hole 12 in position as shown in FIG. 4A.

The fixing rod 7 has two ends which are a first fixing portion 71 and a second fixing portion 72. The first fixing portion 71 may be a polygonal plate protruding from an outer surface of the rod body (not labeled), and the second fixing portion 72 may be an outer thread formed on the outer surface of the rod body, but not limited to this.

The fixing member 8 has a second corresponding fixing portion 81 which is fixed to the second fixing portion 72. The fixing member 8 may be any component capable of being correspondingly fixed to the second fixing portion 72. In the embodiment, a nut is used as an example. Thus, the second corresponding fixing portion 81 may be an inner thread formed on an inner surface of the fixing member 8 to make the second fixing portion 72 be screwed with the second corresponding fixing portion 81.

The fixing portion 71 of the fixing rod 7 is fixed to the first corresponding fixing portion 11 of the carrier 1. The fixing rod 7 projects out from the carrier 1 through the fixing hole 12 to form a projecting portion as shown in FIG. 4A but not labeled (the projecting portion includes the second fixing portion 72). Then the light therapy device 100 is placed in the negative-pressure cup 900 and the projecting portion of the fixing rod 7 projects out from the negative-pressure cup 900 through one of the penetrating holes 9 to expose the second fixing portion 72. Finally, the second corresponding fixing portion 81 of the fixing member 8 is screwed with the second fixing portion 72 of the fixing rod 7 to make both the bottom of the carrier 1 and the bottom of the negative-pressure cup 900 be jointly sandwiched between the first fixing portion 71 and the fixing member 8 for fixation.

Therefore, the light therapy device 100 is disposed in the negative-pressure cup 900, and both the panel 15 used for emitting light and the needle protection cap 5 used for spreading negative ions may face a patient's portion to be relaxed.

While this disclosure has been described by means of specific embodiments, numerous modifications and variations could be made thereto by those skilled in the art without departing from the scope and spirit of this disclosure set forth in the claims.

What is claimed is:

1. A light therapy device, incorporated with a negative-pressure cup, the light therapy device comprising:
a carrier, disposed in the negative-pressure cup, and comprising a panel;
a light therapy assembly, disposed in the carrier;
a negative ion assembly, disposed in the carrier, comprising a releasing needle projecting out from the panel to release negative ions; and
a needle protection cap, covering the panel corresponding to the releasing needle to shield the releasing needle.

2. The light therapy device of claim 1, wherein a needle receiving recess is concavely defined on the panel, a bottom plate is disposed in the needle receiving recess, the negative ion assembly comprises a negative ion releasing head disposed on the panel and comprising the releasing needle, the releasing needle is inserted in the needle receiving recess through the bottom plate, and the needle protection cap covers the panel corresponding to the needle receiving recess.

3. The light therapy device of claim 2, wherein a cap receiving recess is concavely defined on the panel, the bottom plate in the needle receiving recess is defined as a first bottom plate, a second bottom plate is disposed in the cap receiving recess, the needle receiving recess is concavely defined on the second bottom plate, and the needle protection cap is assembled in the cap receiving recess.

4. The light therapy device of claim 2, wherein the panel comprises an inner side and an outer side opposite to each other, the needle receiving recess and a receiving cavity are concavely defined on the panel 15 from the outer side toward the inner side, the receiving cavity and the needle receiving recess are separated by the bottom plate, and the negative ion releasing head is disposed in the receiving cavity.

5. The light therapy device of claim 2, wherein the panel comprises a post extended toward an inward direction of the carrier, a receiving cavity and the needle receiving recess are defined in the post, the receiving cavity and the needle receiving recess are separated by the bottom plate, and the negative ion releasing head is disposed in the receiving cavity.

6. The light therapy device of claim 1, wherein the carrier comprises a light-permeable cover, the light-permeable cover comprises the panel in a light-permeable manner, and the light therapy assembly faces the panel to emit light.

7. The light therapy device of claim 6, wherein the light-permeable cover further comprises a shading plate, the light therapy assembly comprises multiple light emitting elements, the shading plate is assembled on an inner side of the panel and comprises multiple via holes defined thereon, and each light emitting element emits light corresponding to each via hole.

8. The light therapy device of claim 1, wherein the carrier comprises a base and a light-permeable cover assemble to each other, the base comprises an opening, the light-permeable cover covers the base corresponding to the opening, a receiving space is jointly defined between the base and the light-permeable cover, the light therapy assembly and the negative ion assembly are disposed in the receiving space, the light-permeable cover comprises the panel in a light-permeable manner, and the light therapy assembly faces the panel to emit light.

9. The light therapy device of claim 1, further comprising: a fixing rod and a fixing member, wherein a fixing hole is defined on the carrier, one end of the fixing rod is fixed to the carrier, another end of the fixing rod projects out from the carrier through the fixing hole and passes through the negative-pressure cup to be exposed, the fixing member is correspondingly fixed to the another end, and the carrier and the negative-pressure cup are jointly sandwiched between the one end of the fixing rod and the fixing member.

10. The light therapy device of claim 1, wherein the light therapy assembly comprises multiple light emitting elements, each light emitting element comprises a light emitting diode (LED), and the LED comprises a wavelength between 415±10 nm and 830±10 nm.
